(19) United States
(12) Patent Application Publication
(10) Pub. No.: US 2004/0214991 A1
(43) Pub. Date: Oct. 28, 2004

(54) METHOD FOR MEANUFACTURING A COMPOSITE COLLAGEN FILM

(76) Inventors: Jo-Yi Hsiao, Tao-Yuan Hsien (TW); Hsiao-Cheng Yen, Tai-Chung City (TW)

Correspondence Address: NAIPO (NORTH AMERICA INTERNATIONAL PATENT OFFICE), P.O. BOX 506, MERRIFIELD, VA, 22116, US

(21) Appl. No.: 10/249,592

(22) Filed: Apr. 22, 2003

(51) Int. Cl. B05D 3/02; C07K 14/78

(52) U.S. Cl. 530/356; 427/374.1

(57) ABSTRACT

A method for manufacturing a composite collagen film. The method includes providing a collagen film and dipping the collagen film in a solution of collagen and glycosaminoglycans (GAGs). The method further includes sequentially performing a freezing process, a vacuum freezing drying process, a condensing process, and a drying process. Finally, the method comprises using a cross-linking agent for making the collagen film cross link with the collagen and GAGs in the solution so as to produce a composite collagen film.

### BACKGROUND OF INVENTION

1. Field of the Invention

The invention relates to a method for manufacturing a composite collagen film applied to a Guided Tissue Regeneration technology, and more particularly, to a method for manufacturing a composite collagen film with a suturable ability and a biocompatible slow-resorbing property that can be absorbed slowly by an organism.

2. Description of the Prior Art

The Guided Tissue Regeneration (GTR) technology is currently a widely used surgery process. The object of GTR is to restore or regenerate the morphology and functionality of a tissue or an organ having damage caused by illnesses or traumas.

The process of a successful GTR is to make a regenerated tissue repopulate in the original location and space of the damaged tissue. Furthermore, for restoring the morphology and functionality of each kind of different regenerated tissues in a same area, the repopulation of the cells and the following differentiation of the regenerated cells in the area must have an orderly and concerted process. However, when a portion of the living tissue is damaged, an empty space will occur. Therefore the empty space will be filled with the adjacent cell lines with the fastest repopulation rate.

Taking the periodontology as an example, the cells for healing up a periodontal tissue comprise four main resource cells: the epithelium cells and connective tissue cells from the gums, the cells from the bone tissue, and the cells from the periradicular ligament, wherein only the periradicular ligament cells have the tissue regeneration ability. However, the repopulation rate of the epithelium cells is faster than that of the periradicular ligament cells, therefore to restore a damaged periradicular ligament tissue is very difficult.

The GTR is a technology that can make predetermined tissue cells repopulate orderly and concertedly to regenerate a damaged tissue. For the purpose, a barrier is used to be positioned between the predetermined regeneration tissue and the tissue which may interfere with the regeneration of the predetermined tissue until the predetermined tissue has regenerated and been in a maturity state. In the above mentioned example of healing up a periodontal tissue, the GTR is to locate a biomaterial film to prevent the epithelium cells and connective tissue from the gums from connecting with the tooth root so that only the peri-radicular ligament cells is allowed to grow.

There are two main biomaterials are currently used for applying to the GTR. One is un-absorbable polymer film, such as the expanded polytetrafluoroethylene (EPTFE) film and the high-density MEDPOR film. The EPTFE film is un-absorbable, so that the EPTFE film will be very stable during the period of an implanted time, which means the EPTFE can be a good barrier having a high barrier effect. However, the EPTFE film cannot stay in an organism for an unlimited time. Therefore after six to eight weeks from the first surgery of implanting the EPTFE film into the organism, a second surgery is needed to take out the EPTFE film.

Another one is resorbable film comprising the collagen or other polymer, such as the polyglycolates. An extra surgery for removing the barrier is not needed when using the resorbable film as the barrier, because at last the implanted body environment will resorb the resorbable film. The disadvantage is that the resorbable film may not stay completely in the organism for an enough period to guide a successful tissue regeneration.

### SUMMARY OF INVENTION

It is therefore a primary objective of the claimed invention to provide a method for manufacturing a composite collagen film with a suturable ability and a biocompatible slow-resorbing property for applying to the GTR to solve the above-mentioned problem.

In the preferred embodiment according to the claimed invention, the method comprises providing a collagen film, dipping the collagen film in a solution of collagen and glycosaminoglycans (GAGs), sequentially performing a freezing process, a vacuum freezing and drying process, a compressing process, and a cleaning and drying process, and then using a cross-linking agent to make the collagen film link with the collagen and the GAGs in the solution so as to form at least another collagen film on the surface of the collagen film.

It is an advantage of the claimed invention that the method can manufacture a collagen film with a composite structure, so that it will reduce the rate of the barrier being resorbed into the implanted body and extend the time of the barrier staying in the implanted body, and furthermore to increase the barrier of the collagen film. In addition, the collagen film according to the claimed invention further comprises at least a kind of anti-microbial agent, anti-inflammatory agent, or tissue generation guiding factor to release one or several specific agents during different periods of treatment and further increase the effect of the wound healing and tissue regeneration.

These and other objectives of the present invention will no doubt become obvious to those of ordinary skill in the art after reading the following detailed description of the preferred embodiment, which is illustrated in the multiple FIGUREs and drawings.

### BRIEF DESCRIPTION OF DRAWINGS

No drawings are provided.

### DETAILED DESCRIPTION

In the preferred embodiment according to the present invention, the method comprises providing a collagen film. The process of providing the collagen film includes performing an pepsinization treatment by incubating a bovine skin, a tendon, or a placenta with rich collagen, and then solving the solution (1-10 mg/ml) of the purified collagen molecule in a 0.05M of acetic acid solution to bring a reaction at 4° C.

After a period of time, a 0.1 M solution of sodium hydroxide is added to neutralize the acetic acid solution. Then the solution is poured into a mold to keep reacting at 20˜37° C. After that, a piston is used to comprise out the water from the solution until a predetermined thickness of the collagen film is observed. Finally the collagen film is dipped into a dimethylformamide (DMF) solution of the diphenylphosphoryl-azide (DPPA) with a volume concentration of 0.5% to make the fibrils in the collagen film cross-link with each other for increasing the resistance to the enzyme degradation so that the collagen film is gained.

Then a solution of glycosaminoglycans (GAGs) and collagen of Type I is provided, wherein the contain ration of GAGs to the collagen is about 18˜25%. The prepared process of the GAGs and collagen solution comprises preparing a GAGs solution with the weight concentration of 0.5˜2%, solving the collagen fibrils in a light acidic solution, such as a 0.1 M of an acetic acid solution, to gain a collagen solution with the same concentration, then mixing the two solutions, and finally neutralizing the solution with a sodium hydroxide solution to adjust the pH value of the mixed solution to about 6.5˜8.

After that, the collagen film is dipped in the mixed solution and a freezing process is performed by using liquid nitrogen at −70° C. The purpose of the freezing process is to form a plurality of micro-pores in the collagen film so as that the H₂O molecules and nutrition molecules can pass through the micro-pores to provide the nutrient needed by the regeneration when the produced collagen film serving as a barrier is positioned in an area of damaged tissue.

Sequentially a vacuum freezing and drying process and a compressing process are performed for about 18 hours to remove H₂O for observing a collagen film with a determined thickness. Then a natural cross-link agent, a genipin or a glutaraldehyde, is used to make the collagen film adsorb the collagen and the glycosaminoglycans in the mixed solution so as to gain a composite collagen film with a composite structure. Then a further treatment is provided to clean and dry the composite collagen film. And the mentioned above steps may be repeated to produce a composite collagen film with a multi composite structure according to the clinical needs.

According to the present invention, the method can manufacture a composite collagen film with a biocompatible slow-resorbing property to increase the barrier effect. The method further comprises adding one or several kinds of anti-microbial agent, anti-inflammatory agent, or tissue generation guiding factors to immobilize the additives into different structure areas of the composite collagen film.

For a composite collagen film with a multi composite structure, a cross-link agent with different medical agents may be used to make the composite collagen film have at least one cross-link reaction to form a multi composite collagen film with different medical agents in different structure areas of the multi composite collagen film. The medical agents immobilized in the multi composite collagen film may be anti-microbial agents, anti-inflammatory agents, and tissue generation guiding factors. Therefore when the multi composite collagen film is degraded and resorbed gradually, the specific agents may be released according to the time and the degrading state of each structure area of the composite collagen film to increase the effect of wound healing and tissue regeneration.

In contrast to the prior art, the present invention method can manufacture a composite collagen film with a slow-resorbing rate and extend the time of the composite collagen film staying in an implanted body, so that the barrier effect will be increased and one or several specific agents may be released during different periods of treatment to increase the effect of the wound healing and tissue regeneration.

Those skill in the art will readily observe that numerous modifications and alterations of the device may be made while retaining the teachings of the invention. Accordingly, the above disclosure should be construed as limited only by the metes and bound of the appended claims.

What is claimed is:

1. A method for manufacturing a composite collagen film, the method comprising following steps:
(a) providing a collagen film;
(b) providing a solution of collagen and glycosaminoglycans (GAGs), and dipping the collagen film in the solution;
(c) performing a freezing process;
(d) performing a vacuum freezing and drying process;
(e) performing a compressing process; and
(f) performing a cross linking process by using a cross-linking agent to make the collagen film cross link with the collagen and GAGs in the solution so as to form a composite collagen film on the surface of the collagen film.

2. The method of claim 1 further comprising to repeat the steps (b) to (f) at least once to form the composite collagen film on the surface of the collagen film so as to make the composite collagen film have a multi composite structure.

3. The method of claim 1, wherein the process of providing the collagen film comprises following steps:
(g) putting a collagen in a pepsin and performing a first incubating process;
(h) solving the incubated collagen in a solvent;
(i) introducing the solved collagen into a mold and performing a second incubating process to make the solved collagen become a film;
(j) compressing the collagen film to a predetermined thickness; and
(k) reacting the compressed collagen film with a cross-link agent to make the fibrils in the compressed collagen film cross link with each other so as to form a stable matrix of the collagen film.

4. The method of claim 3, wherein the collagen is a collagen of type I.

5. The method of claim 3, wherein the solvent is an acetic acid.

6. The method of claim 1, wherein the freezing process is performed by using a liquid nitrogen to lower a temperature to −180° C.

7. The method of claim 6, wherein the freezing process is used for forming a plurality of micro-pores in the collagen film.

8. The method of claim 1, wherein the process time of the vacuum freezing drying process is about 18 hours.

9. The method of claim 1, wherein the cross-link agent is a glutaraldehyde.

10. The method of claim 1, wherein the cross-link agent further comprises at least an anti-microbial agent, an anti-inflammatory agent, or a tissue generation guiding factor, and the anti-microbial agent, the anti-inflammatory agent, or the tissue generation guiding factor are capable of being immobilized in the composite collagen film by the cross-link agent.

11. The method of claim 10, wherein the anti-microbial agent, the anti-inflammatory agent, and the tissue generation guiding factor are capable of being immobilized in the composite collagen film with the multi composite structure by different cross-link agents in different structure areas of the composite collagen film respectively.

12. The method of claim 11, wherein the anti-microbial agent, the anti-inflammatory agent, or the tissue generation guiding factor are capable of being gradually released from the composite collagen film into an implanted body environment according to the decomposed period of the different structure areas of the composite collagen film with the multi structure.

13. The method of claim 1, wherein the composite collagen film serves as a guided tissue regeneration (GTR) film.

14. The method of claim 1 further comprising a cleaning and drying process after step (e).
